(19) United States
(12) Patent Application Publication
(10) Pub. No.: US 2008/0194678 A1
(43) Pub. Date: Aug. 14, 2008

(54) Process To Obtain Synthetic And Semi-Synthetic Lignan Derivatives, Their Antiparasitic Activities And Corresponding Pharmaceutical Formulations, Including The Therapeutic Method Using Said Lignan For The Treatment Of Parasitosis

(75) Inventors: Marcio Luis Andrade e Silva, Franca (BR); Rosangela da Silva, Jaboticabal (BR); Vanderlei Rodrigues, Ribeirao Preto (BR); Olavo dos Santos Pereira, Ribeirao Preto (BR); Ademar Alves da Silva Filho, Liberdade (BR); Paulo Marcos Donate, Ribeirao Preto (BR); Sergio Albuquerque, Ribeirao Preto (BR); Jairo Kenupp Bastos, Ribeirao Preto (BR)

Correspondence Address: VOLPE AND KOENIG, P.C., UNITED PLAZA, SUITE 1600, 30 SOUTH 17TH STREET, PHILADELPHIA, PA, 19103, US

(73) Assignee: Fundacao de Amparo a Pesquissa do Estado de Aao Pa, Sao Paulo (BR)

(21) Appl. No.: 11/995,789

(22) PCT Filed: Jul. 14, 2006
(86) PCT No.: PCT/BR2006/000144

§ 371 Date: Apr. 16, 2008

(30) Foreign Application Priority Data
Jul. 15, 2005 (BR) PI 0503951-7

(51) Int. Cl. A61K 31/357; C07D 317/46; C07D 307/02; A61P 33/12; A61K 31/34; C07D 317/70

(52) U.S. Cl. 514/464; 549/435; 549/502; 549/432; 514/461; 514/463

(57) ABSTRACT

A process to obtain synthetic and semi-synthetic derivatives of lignans, especially dibenzylbutyrolactonic, tetrahydrofuranic, aryltetralynic, furofuranic and dibenzocyclooctanic lignans obtained by means of partial synthesis and/or full synthesis or also by isolation from plant extracts. It refers to a process to obtain synthetic and semi-synthetic derivatives of (−)-cubebin, such as: (−)-O-acetylcubebin; (−)-O-methylcubebin; (−)-O—N,N-(dimethylamino-ethyl)-cubebin; (−)-hinokinin; (−)-6,6′-dinitroinokinine; (−)-O-benzylcubebin; (−)-6,6′-diaminoinokinin, (−)-6,6′-dinitroinokinin, as well as to obtain dibenzocyclooctanic lignans from dibenzylbutyrolactoinic lignans by means of structural modifications in the positions 7, 7′, 8, 8′, 9′ and in the aromatic rings (introduction and/or substitution of functional groups such as: —OH, —CO₂H, —CO₂CH₃, —NO₂, —NH₂, —OCH₃, —OAc, —SO₂CH₃, —SO₂NH₂, prenyl and halogens) is provided. A therapeutic method using the derivatives is also provided.

### FIELD OF THE INVENTION

The invention refers to a process to obtain lignan derivatives by full or partial synthesis, especially dibenzylbutyrolactonic, tetrahydrofuranic, furofuranic, dibenzocyclooctanic and aryltetralynic lignans, which may also be isolated from natural sources, including plant species Zanthoxylum naranjillo, Piper cubeba and Nectandra megapotamica. In vitro and in vivo studies have revealed the capacity of some of these substances to promote the separation of parasite couples, which causes the suspension of egglaying by the parasite. Male and female adult parasites were killed by some of these substances due to the destruction of their membrane. The invention also refers to a therapeutic method, since the described substances are used to manufacture medicine that presents antiparasitic activity for the treatment of schistosomosis and other parasitosis.

More specifically, it refers to a process to obtain synthetic and semi-synthetic derivatives of dibenzylbutyrolactonic lignans, methylpluviatolide (1) and (−)-cubebine (2), such as: (−)-hinokinin(3), (−)-O-acetylcubebin (4); (−)-O—N,N-(dimethylamino-ethyl)-cubebin (5); (−)-O-methylcubebin (6), (−)-O-benzylcubebin(7); (−)-6,6′-dinitroinokinin(8), (−)-6,6′-dinitrocubebin (8a), (−)-6,6′-diaminoinokinin (9), aryltetralynic and dibenzocyclooctanic derivatives, as well as sesamin (10) and their derivatives to be used in the manufacture of medicine that presents antiparasitic activity, especially against parasites of the genera Schistosoma and nematodes. The present invention also refers to the process to obtain substances galgravin (11), veragensin (12), nectandrin A (13) and nectandrine B (14) from Nectandra megapotamica, as well as their synthetic and semi-synthetic derivatives with substituents on the aromatic rings.

### BACKGROUND

Bioresearch of new active principles from higher plants to be used as prototypes for the development of new medicines has been a constant practice by researchers in the last decades, which is based on random and rational screenings. In the last twenty years, about 40% of new medicines introduced into the market have natural origin or were obtained from natural prototypes.

The search for new therapeutic alternatives that are safer and more effective is extremely important to overcome currently existing problems. The lignans described here present excellent antiparasitic activities and with practically no side effects for their use.

Many classes of different natural products have been used to synthesize new pharmaceuticals, such as terpene derivatives used as raw materials for the synthesis of artemisin and sesquiterpene derivative with important anti-malaria activities. The classes of lignans and neolignans, which present high potential, since antitumoral, antiviral, anti-inflammatory and anti-Chagas activities have already been disclosed, among others, bear high potential for the development of new medicines.

According to the World Health Organization (WHO), in tropical and subtropical areas, schistosomiasis is only surpassed by malaria in terms of social/economic importance and public health. The disease is constantly present in over 74 countries (most of them underdeveloped), between 500 and 600 million people are exposed to infection and more than 200 million are infected each year.

Schistosomiasis (form adopted by the International Disease Classification) is also known as bilharziosis or schistosomosis. It is caused by worms of the genus Schistosoma, which infest mainly the mesenteric veins of the portal system, where they attach by means of suckers and start laying eggs. Among various species, we emphasize S. haematobium (more common in Africa and the Mediterranean), S. japonicum (more common in South East Asia) and S. mansoni (especially present in American countries, such as Brazil), which is described as follows. The worm belongs to the family of trematodes, has separate sexes and may reach 1 cm of length.

The water is the means used by Schistosoma to infect men (main host) and the mollusk of the genus Biomphalaria (intermediate host). The evolution cycle of schistosomiasis starts when feces from infested individuals, containing eggs from the parasite, get in contact with water. The eggs hatch and release the first larval form of Schistosoma, known as miracidium. The larva needs appropriate environmental conditions to survive, which destroys the myth that schistosomiasis only occurs in polluted waters.

As soon as it leaves the egg, the miracidium searches and penetrates the mollusk where, during 20 or 30 days, it will multiply and transform into another larva, known as cercaria. One mollusk can release thousands of cercariae in a single day, starting the second phase of the cycle. Usually, cercariae are released between 11:00 a.m. and 5:00 p.m., rarely at night, and they can only survive a few hours. Once in the water, the cercaria swims in search of its definitive host.

After penetrating the human body, the cercaria migrates to the blood or lymph stream. After one day of infection, larvae can be found in the lungs and after nine days they migrate to the liver, where they feed on blood and start their maturation. After twenty days, adult worms start to breed and after seven days the female is already laying the first eggs. On average, only after the fortieth day of infection is it possible to find eggs of S. mansoni in patient's feces.

Fever, headache, chills, sweats, weakness, lack of appetite, muscular aches, coughs and diarrhea are the symptoms of schistosomiasis in the acute stage. Liver and spleen also increase due to inflammation caused by the presence of the worm and its eggs. If not treated, the disease may evolve to its chronic form, where diarrhea is more and more frequent, alternating with constipation and the feces may contain blood. The patient has dizziness, itching of the anus, palpitations, impotence, weight loss and enlargement and hardening of the liver. In this stage, the appearance of the patient becomes characteristic: weak, with an enormous abdomen, which gives the disease its popular name “water belly”.

The treatment is made by chemotherapy, especially by the administration of drugs such as oxamniquin or praziquantel. Oxamniquin is effective in about 80% of the cases in adults and 65% of the cases in children, but it presents side effects, such as: cephalgia, sleepiness, nausea, tremors and rarely convulsions. Praziquantel presents healing percentages similar to oxamniquine, with the following side effects: abdominal pain, diarrhea, fever and urticaria-shaped reactions.

### SUMMARY

During our research, we observed that isolated lignans of Zanthoxylum naranjillo (Rutaceae), such as (−)-methylpluviatolide (1) and (−)-cubebin (2), as well as synthetic and semi-synthetic derivatives (−)-hinokinin (3), (−)-O-acetylcubebin (4), (−)-O—(N,N-dimethylaminoethyl)-cubebin (5), (−)-O-methylcubebin (6), (−)-O-benzylcubebin(7), (−)-6,6′-dinitroinokinin (8), (−)-6,6′-dinitrocubebin (8a), (−)-6,6′-diaminonokinin (9) and their aryltetralynic derivatives (1a, 1b, 1c, 2a, 2b, 2c, 2d, 2e, 2f, 2g, 2h), whose chemical structures are shown by Schemes 1 and 2, bear significant schistosomicidal activity.

Also, furofuranic lignans, such as (+)-sesamine (10) and piperitol (11), pluviatilol (12) and xantoxilol (13) derivatives, are potentially active—Scheme 3. Tetrahydrofuranic lignans from Nectandra megapotamica (Lauraceae), such as: machilin (14a), galgravin (14b), nectandrin A (14c), nectandrin B (14d), caloptin (16a), veragensin (15b), aristolignine (15c), ganschisandrin (16a), nectandrin D (16b), nectandrin E (16c) and their synthetic derivatives also presented significant schistosomicidal activity—Scheme 4, as well as tetrahydrofuranic lignan magnosalicin (17) and its synthetic derivatives and diaestereoisomers (18, 19, 20 and 21)-Scheme 5. Also, dibenzocyclooctanic lignans: stegan (22), steganon (23), steganacin (24), steganol (25) and their synthetic derivatives—Scheme 6 are also potentially active.

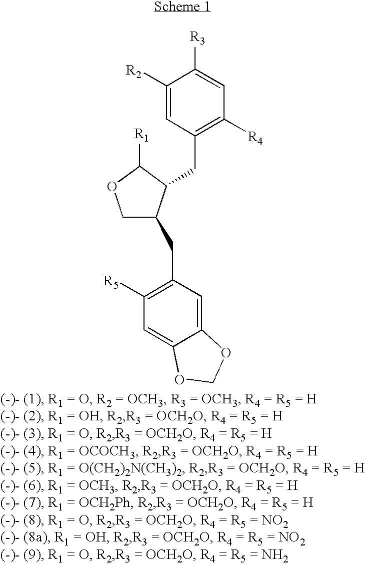

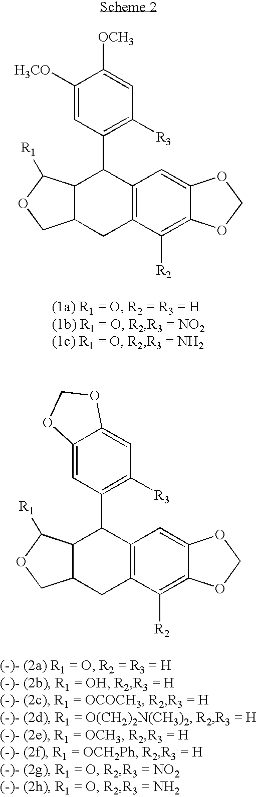

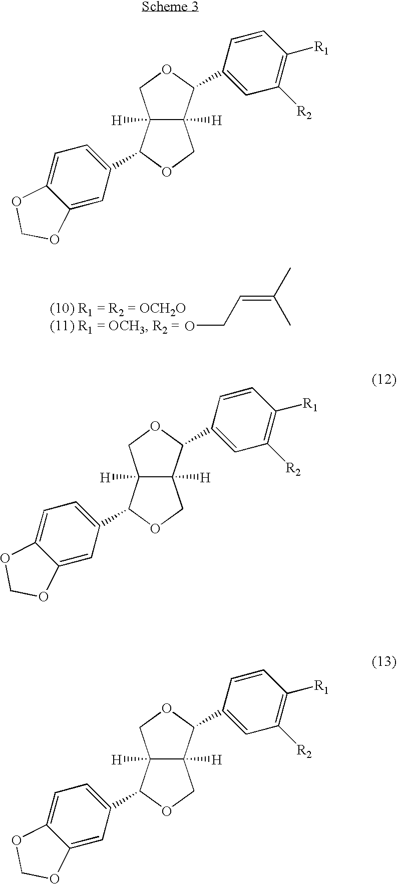

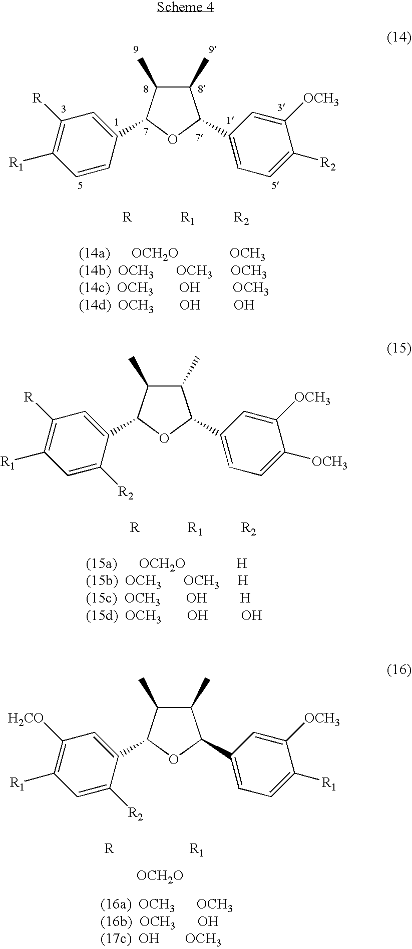

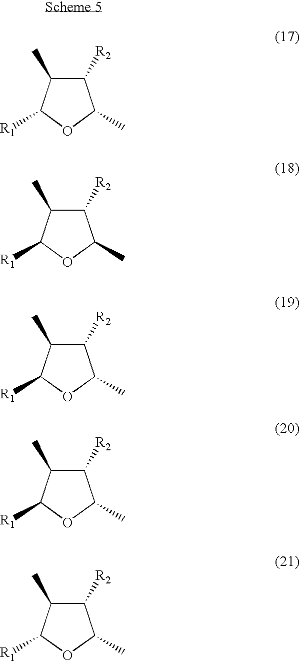

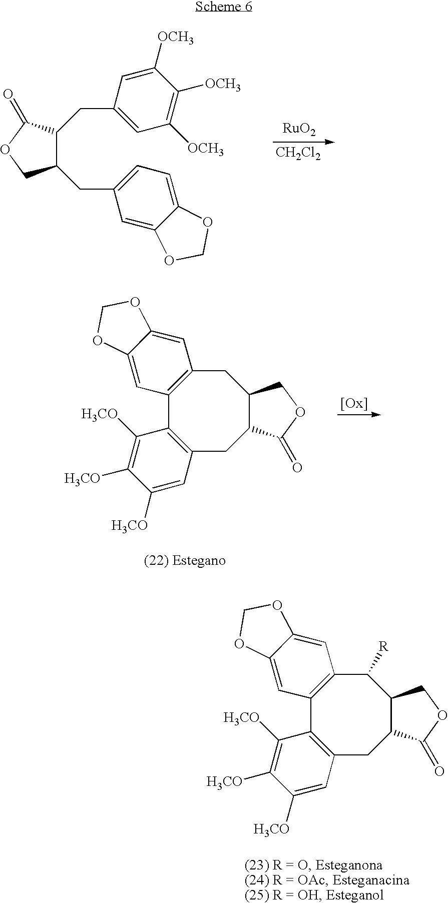

Schistosoma mansoni is a trematode of the class Digenea that has separate sexes with notable sexual dimorphism. Its geographical distribution is conditioned to the presence of mollusks of the genus Biomphalaria in fresh water, which act as intermediate hosts. Parasitosis caused by S. mansoni is called mansonic schistosomosis and, only in Brazil, it is estimated that the parasite infects about 2.5 million people, who in many cases present severe organic deficiencies. Approximately 25 million people live with the potential risk of infection by the parasitosis, making this disease one of the serious public health problems in Brazil.

The biology of Schistosoma presents a complex development plan, involving the exchange from a water environment to another in the invertebrate intermediate host and ending in a habitat in the definitive vertebrate host.

The current choice of medicine for individuals affected by schistosomosis is praziquantel, which has limited action in the evolutionary stage of adult worm of the parasite S. mansoni, and strains resistant to the medicine have already been found.

The use of substances derived from “(−)-cubebin (1)” has shown high potential of action against the parasite at issue.

The purpose of the invention herein proposed is to obtain semi-synthetic derivatives of dibenzylbutyrolactonic lignans methylpluviatolide (1) and cubebine (2): (−)-hinokinine (3), (−)—O-acetylcubebin (4), (−)—O—N,N-(dimethylamino-ethyl)-cubebin (5), (−)—O-methylcubebin (6), (−)—O-benzylcubebin(7), (−)-6,6′-diaminoinokinin (8), (−)-6,6′-dinitroinokinin (9)-Scheme 1 and their aryltetralynic derivatives (1a-1c) and (2a-2h)—Scheme 2.

### Obtaining Derivatives

The synthetic route adopted has already been used by various authors to obtain numerous lignan-lactones and is shown in Scheme 7 as follows:

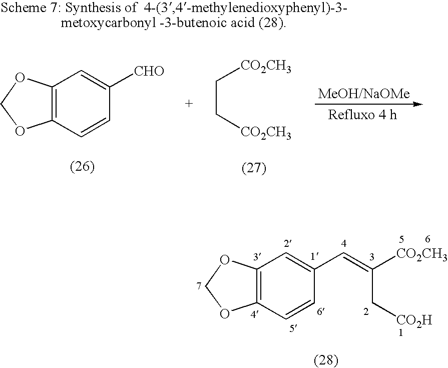

The Stobbe condensation between piperonal (26) and methyl succinate (27) gives unsaturated acid (28)—Scheme 7. This acid, when submitted to catalytic hydrogenation at 20H₂ atmospheres, gives acid (29)-Scheme 8.

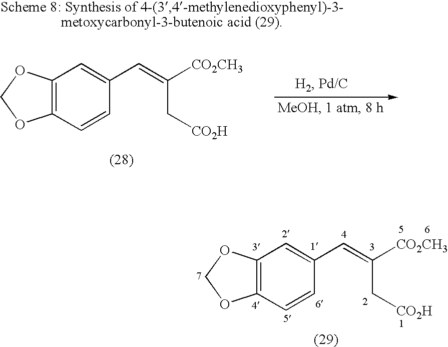

The treatment of 4-(3′,4′-methylenedioxyphenyl)-3-metoxycarbonyl-3-butanoic acid (29) with KOH in ethanol gives potassium salt which, in reaction with Ca(BH4)2 generated in situ, reduces ester grouping to alcohol and, after acid treatment, yields lactone (30)—Scheme 9.

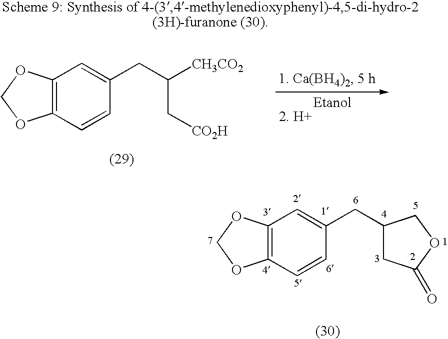

The lactone 4-(3′-4′-methylenedioxyphenyl)-4,5-di-hydro-2 (3H)-furanone (30) submitted to treatment with two LDA equivalents gives the respective enolate which, in reaction with pirenonal and/or veratraldehyde, yields compounds (31) and (32), respectively.

The reaction between the lactone 4-(3′,4′-methylenedioxyphenyl)-4,5-di-hydro-2 (3H)-furanone (30) with LDA/aromatic aldehyde solely provides trans product, related to protons 8 and 8′ (lactonic ring) in the form of two diaestereoisomers each as a pair of enantiomers, as we can see in Scheme 10 as follows.

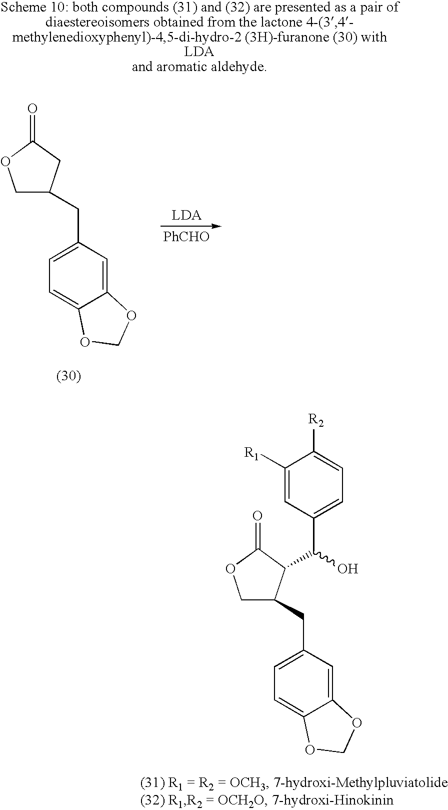

From the hydrogenolysis reaction of the compounds (31) and (32), as shown in Scheme 11, it is possible to obtain lignans dibenzylbutyrolactones methylpluviatolide (1) and hinokinin (3), respectively. The same intermediates in reaction with trifluoroacetic acid may also yield aryltetralynic lignan lactones, whose basic skeleton is analogous to podophylotoxin, widely known for the biological properties of its derivatives.

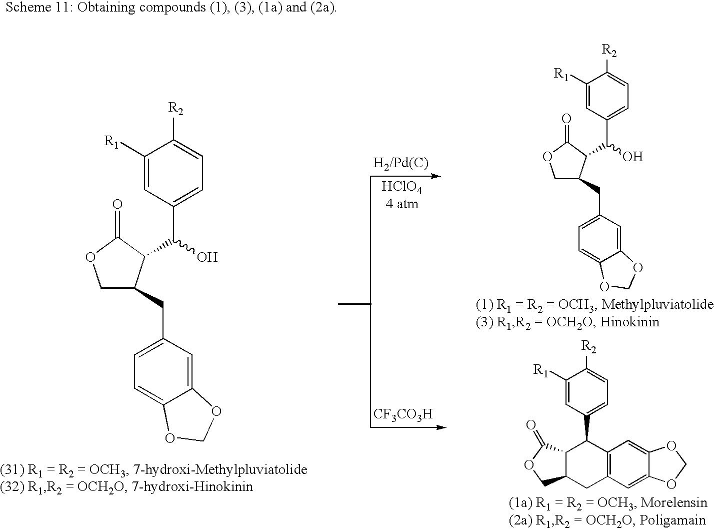

The treatment of the intermediary (31) with CF₃CO₂H/CH₂Cl₂ gives morelensine (1a) lignan lactone aryltetralin derived from methylpluviatolide (1). The same reaction with the intermediary (32) provides polygamain (2a) lignan lactone aryltetralin derived from cubebin (2). The compound (2b) is obtained by the reduction reaction between (2a) and DIBAL. Compounds (2c) to (2f) are obtained by the reaction between (2b) and, respectively: acetic anhydride/py, dimethylethylamine chloride/NaH, methyl iodide/NaH and benzyl bromide/NaH—Scheme 12.

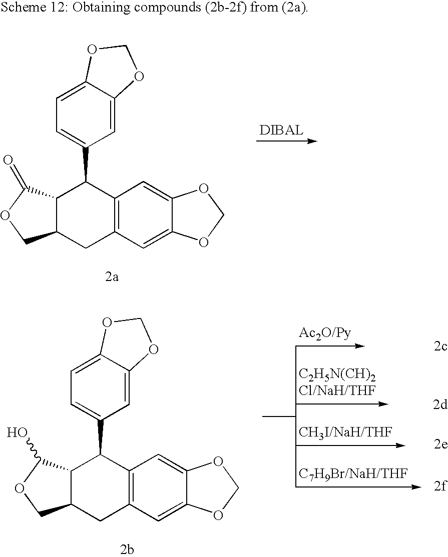

The derivatives obtained by full synthesis are purified by silica gel column chromatography and after that by chiral liquid chromatography to obtain enantiomers + and −.

From (−)-cubebin (2) extracted from Zanthoxyllum naranjillo, the derivatives (−)-hinokinin (3), (−)-O-acetylcubebin (4), (−)-O—N,N-(dimethylamino-ethyl)-cubebin (5), (−)-O-methylcubebin (6), (−)-O-benzylcubebin(7) are obtained and, from (−)-hinokinin (3), the derivatives (−)-6,6′-dinitronokinin (8), (−)-6,6′-dinitrocubebin (8a) and (−)-6,6′-diaminoinokinin (9) are obtained by using, respectively, the following reaction conditions: PCC/CH2Cl2, acetic anhydride/py, dimethylethylamine chloride/NaH, methyl iodide/NaH, benzyl bromide/NaH for the derivatives directly obtained from cubebine—Scheme 13 and HNO3/CHCl3, DIBAL and H2Pd/C, 20 atm. for the derivatives obtained from (−)-hnokinin (3)—Scheme 14.

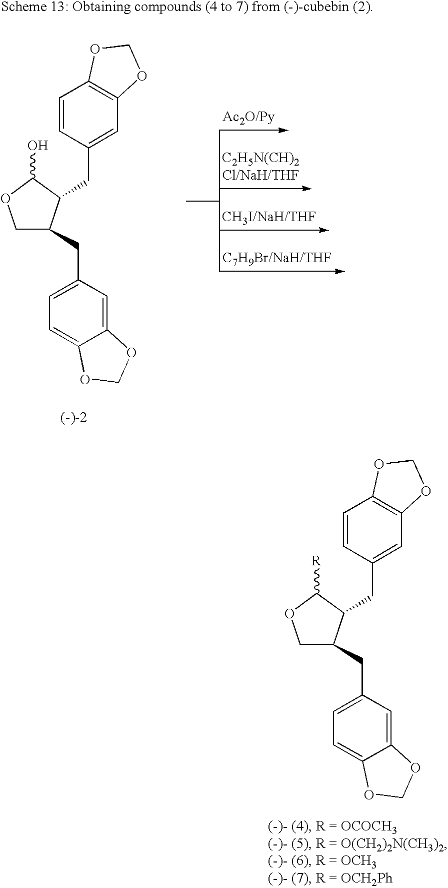

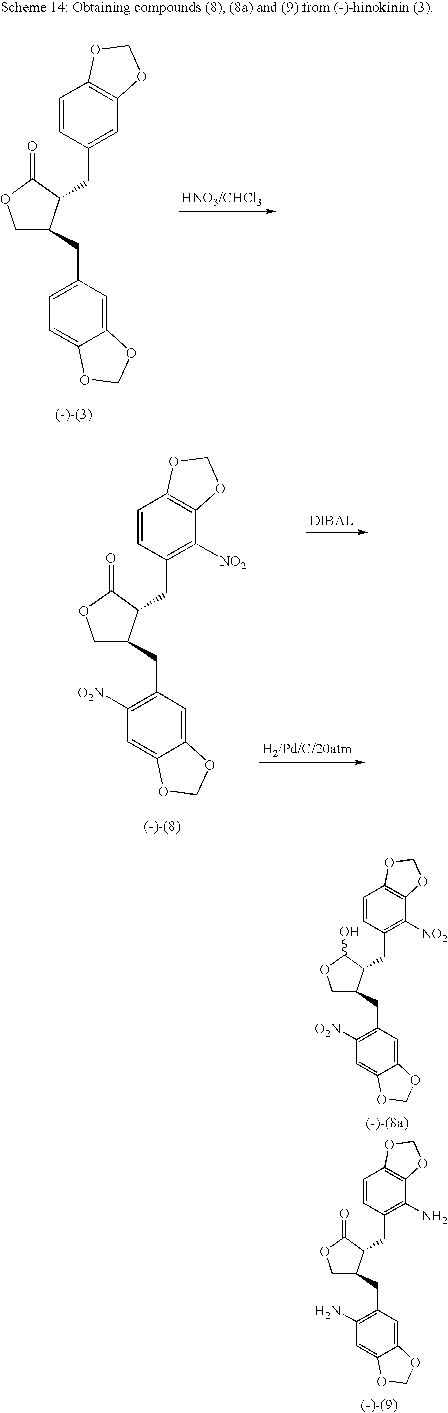

Aryltetralynic lignans—Scheme 2, and tetrahydrofuranic lignans—Scheme 4, machiline (14a), galgravin (14b), nectandrin A (14c), nectandrin B (14d), caloptin (15a), veragensin (15b), aristolignin (15c), ganschisandrin (16a), their synthetic derivatives and dibenzocyclooctanic ligans stegan (22), steganon (23), steganacin (24), steganol (25)—Scheme 6, by the processes herein described to be used in the therapeutics of schistosomosis.

From precursors that belong to the group of dibenzylbutyrolactonic lignans, we obtain the dibenzocyclooctanic lignans using rhutenium oxide (RuO₂)—Scheme 15.

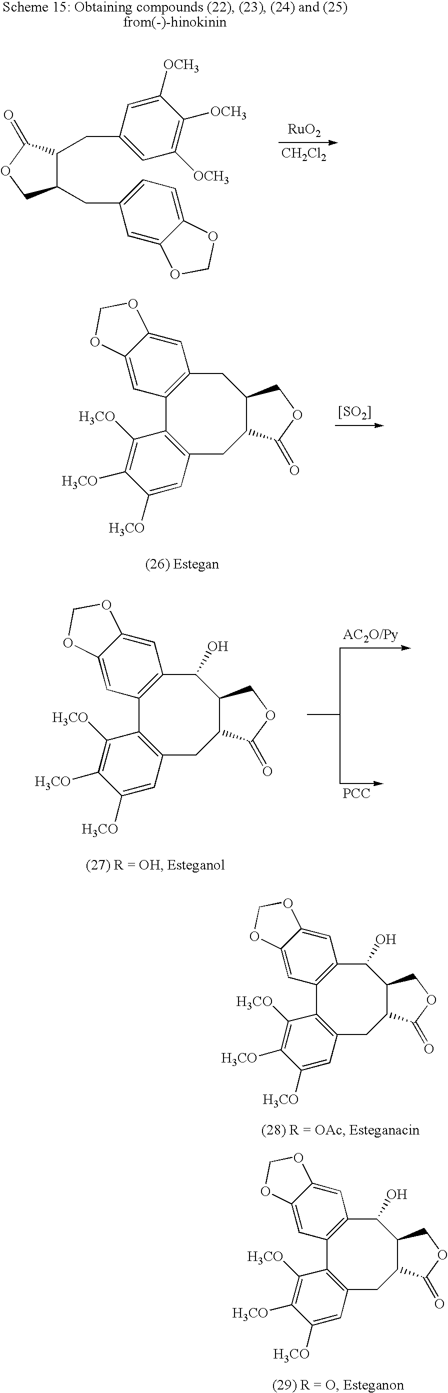

The derivatives from furofuranic, aryltetralynic, tetrahydrofuranic, dibenzocyclooctanic and dibenzylbutyrolactonic lignans are active in concentrations between 3.5 μg/ml and 50 μg/ml.

### BRIEF DESCRIPTION OF THE DRAWINGS

In order to better illustrate and enhance comprehension of the invention proposed, the following figures and schemes are presented:

FIG. 1—effect of the drugs (−)-hinokinin (3) and (−)-O—(N,N-dimethylamino-ethyl) cubebin (4), in concentrations of 14, 7.5 and 3.5 μg/ml, under the parasite Schistosoma mansoni's egglaying during 5 days of culture.

FIG. 2—effect of the drug (−)-6,6-dinitroinokinin (8), in concentrations of 14, 7.5 and 3.5 μg/ml, under the parasite Schistosoma mansoni's egglaying during 5 days of culture.

FIG. 3—effect of the drug (−)—O-methylcubebin (5), in concentrations of 14, 7.5 and 3.5 μg/ml, under the parasite Schistosoma mansoni's egglaying during 5 days of culture

FIG. 4—parasite Schistosoma mansoni (evolutive stage of adult worm) cultivated in vitro in RPMI 1640 medium, in 5% CO2 atmosphere at 37° C. for 24 hours, in the absence and presence of the drug (−)-6,6-dinitroinokinin (8). A and C, male and female parasites, respectively, cultivated in the absence of substances. B and D, male and female parasite, respectively, cultivated in the presence of the Drug (the same effect was noticed in concentrations of 14.0, 7.0 and 3.0 μg/ml) Inverted light microscope, 4× magnification.

FIG. 5—parasite Schistosoma mansoni (evolutive stage of adult worm) cultivated in vitro in RPMI 1640 medium, in 5% CO₂ atmosphere at 37° C. for 24 hours, in the absence of the substances. B and D, male and female parasites, respectively, cultured in the presence of the substances O-methylcubebine (5) and (−)-6,6′-dinitronokinin (8) (the same effect was noticed in concentrations of 14.0, 7.0 and 3.0 μg/ml). Inverted light microscope, 10× magnification.

FIG. 6—parasite Schistosoma mansoni (evolutive stage of adult worm) cultured in vitro in RPMI 1640 medium, in 5% CO₂ atmosphere at 37° C. for 24 hours, in the absence and presence of the substances O-methylcubebine (5) and (−)-6,6-dinitroinokinin (8). A and C, male and female parasites, respectively, cultivated in the absence of the substances. B and D, male and female parasite, respectively, cultivated in the presence of the substances (the same effect was noticed in concentrations of 14.0, 7.0 and 3.0 μg/ml). Inverted light microscope, 20× magnification.

FIG. 7—parasite Schistosoma mansoni (evolutive stage of adult worm) cultivated in vitro in RPMI 1640 medium, in 5% CO₂ atmosphere at 37° C. for 24 hours, in the absence and presence of the substances (−)—O-methylcubebin (5) and (−)-6,6-dinitroinokinin (8). A and C, male and female parasites, respectively, cultivated in the absence of the substances. B and D, male and female parasites, respectively, cultivated in the presence of the substances (the same effect was noticed in concentrations of 14.0, 7.0 and 3.0 μg/ml). Inverted light microscope, 40× magnification.

FIG. 8—graph with the number of recovered worms vs. effect of the drug. Couples of S. mansoni recovered from BALB/c mice by perfusion of the door-kidney system. Five mice, after 40 days of infection (approximately 200 cercariae), were submitted to test with a single dose of the drug dinitrocubebine (10 mg/kg of weight). In the group untreated (negative control treated only with a 5% dimethyl sulfoxide solution in water), 40 couples/animal were recovered. In the treated group, only 5 couples were recovered. These results show that, in a single dose, dinitroinokinin presented around 90% of efficacy.

FIG. 9—graph with the number of recovered worms vs. effect of the drug. Couples of S. mansoni recovered from BALB/c mice by perfusion of the door-kidney system. Five mice, after 40 days of infection (approximately 200 cercariae), were submitted to test with a single dose of the drug methylcubebin (10 mg/kg of weight). In the untreated group (negative control treated only with a 5% dimethyl sulfoxide solution in water), 40 couples/animal were recovered. In the treated group, only five couples were recovered. These results show that, in a single dose, methylcubebine presented around 93% efficacy.

### DETAILED DESCRIPTION OF THE INVENTION

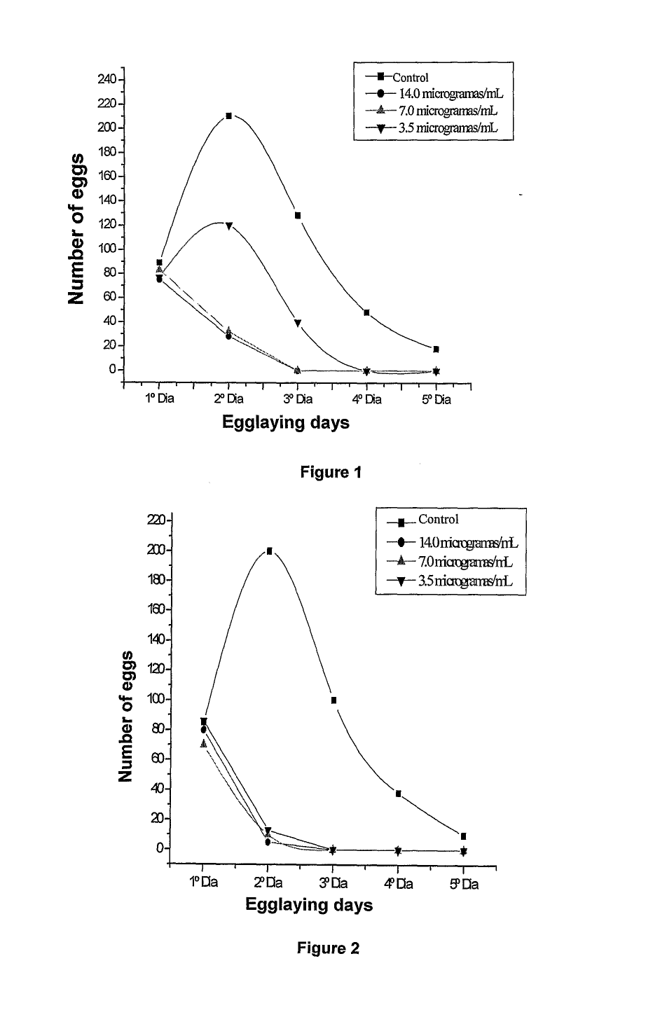

FIG. 1 shows the actions of the substances (−)-hinokinin (3) and (−)-O—N,N-(dimethylamino-ethyl)-cubebin (5), which presented a very similar performance. Two hours after the addition of substances 3 and 5 in concentrations of 14.0 and 7.0 μg/ml, considerable increase in the motility of parasite couples was noticed, ending up with their separation after 18 hours. The same substances, in concentration of 3.5 μg/ml, after 24 hours of culture, caused an increase in the motility of couples and considerable reduction of egglaying, and separation of the couples was observed after 36 hours. The worms were analyzed for 5 days and no apparent change in tegument or death of parasites was observed.

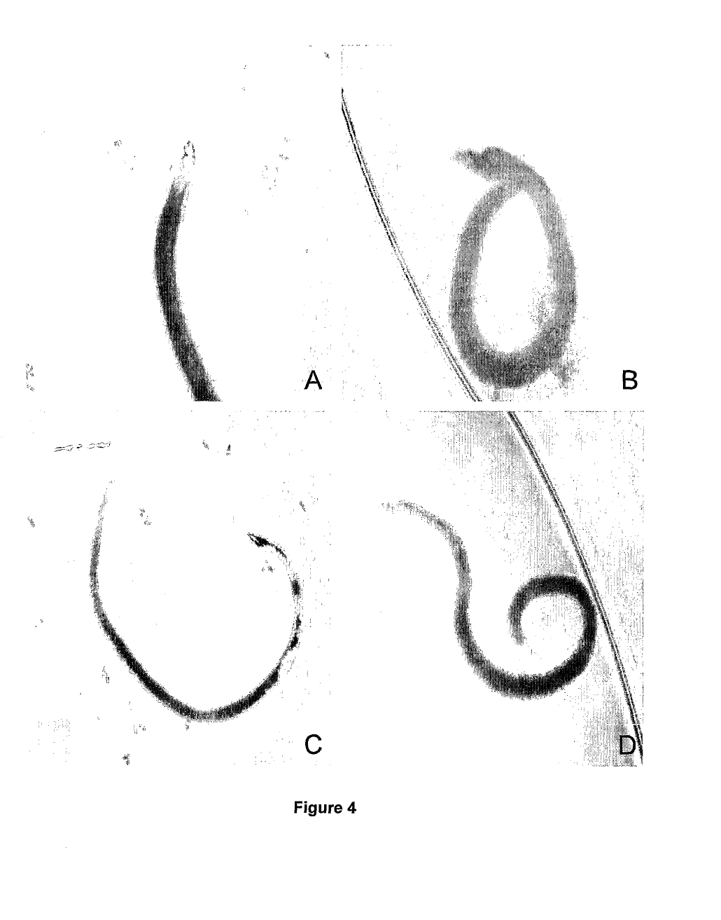

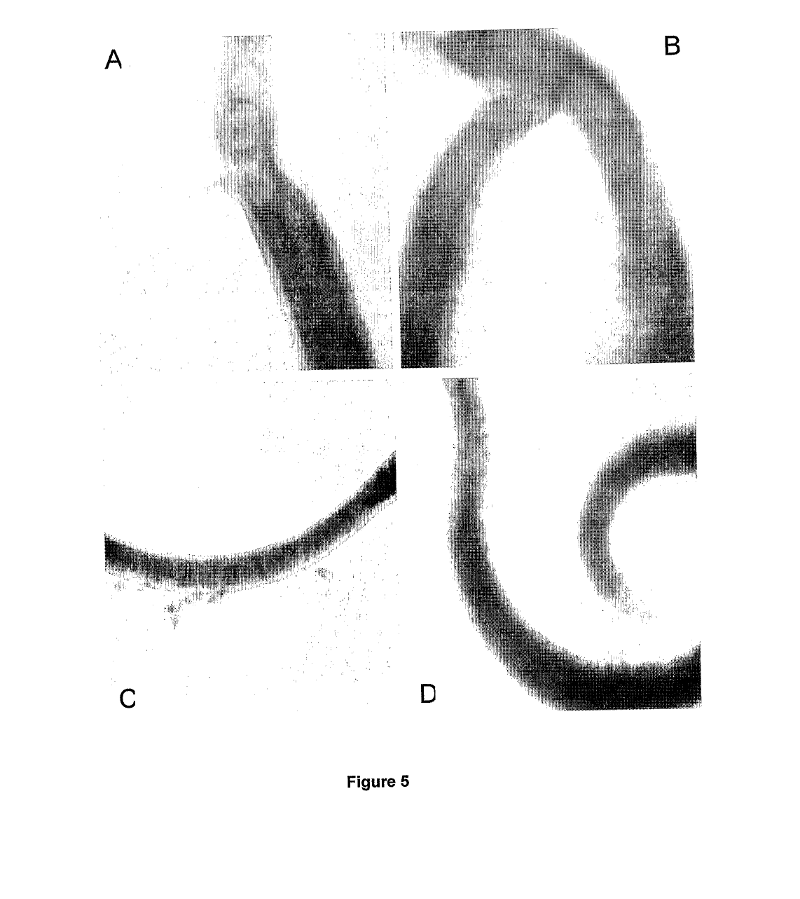

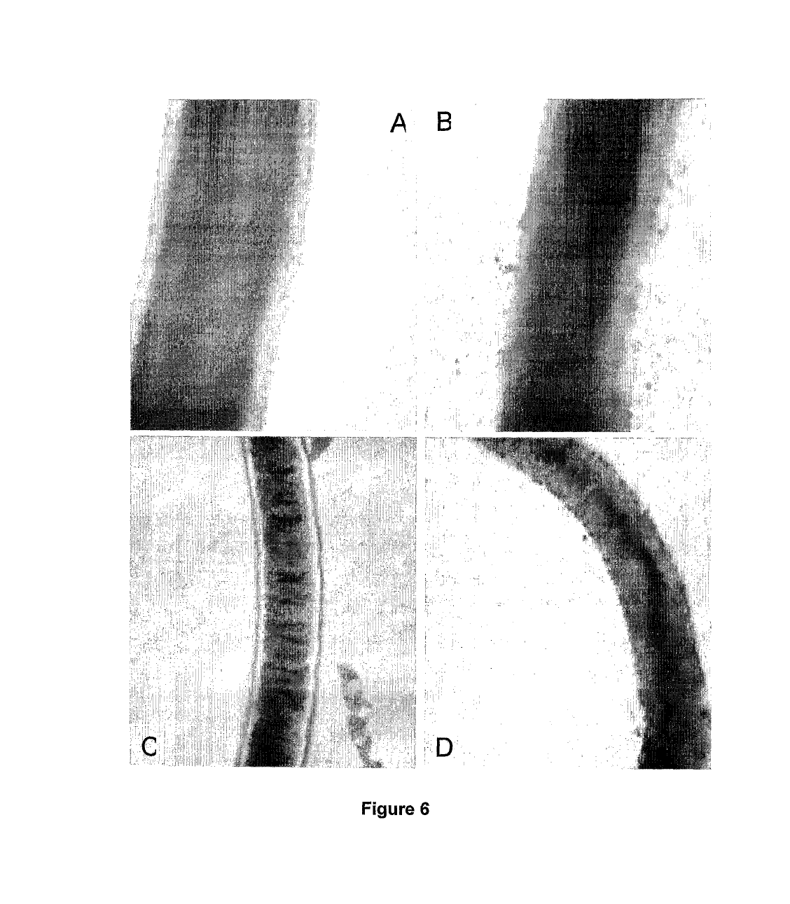

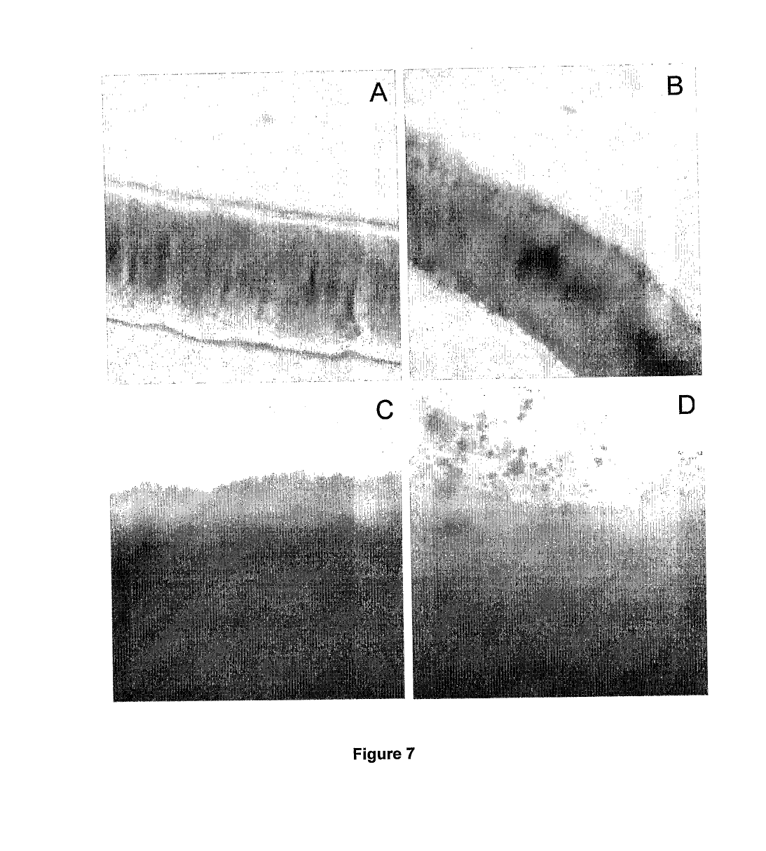

The couples incubated in the presence of the substance (−)-6,6-dinitrohynoquinine (8) in 14, 7.5 and 3.5 μg/ml-1 concentrations, had considerable increase in motility in the first hour of culture, and the separation of the parasites was observed after 12 hours, followed by interruption in egglaying, as shown in FIG. 2. The effect of said substance was extremely pronounced since, besides causing the separation of couples, caused strong tegument lesions in both male and female parasites and their death after 20, 24 and 36 hours, in the presence of the substance in concentrations of 14.0, 7.0 and 3.5 μg/ml-1, respectively (FIGS. 4, 5, 6 and 7).

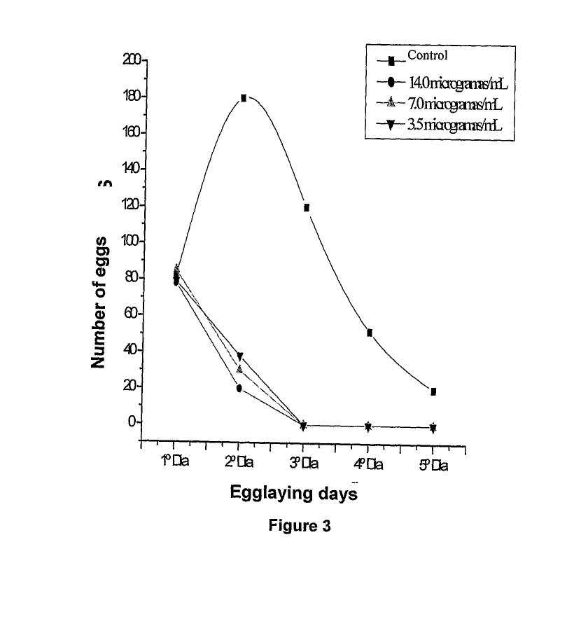

FIG. 3 shows that the action of the substance (−)—O-methylcubebin (6) was very similar to (−)-6,6-dinitroinokinin (8), since it increased the motility of couples after the first hour of culture and their separation during the first 12 hours, but did not produce cutaneous lesions, nor the death of the parasites.

As shown in FIGS. 1 to 7, (−)-6,6-dinitroinokinin (8), in all tested concentrations (14.0, 7.0 and 3.5 μg/ml), caused very evident tegument lesions in male and female Schistosoma mansoni parasites, causing their death.

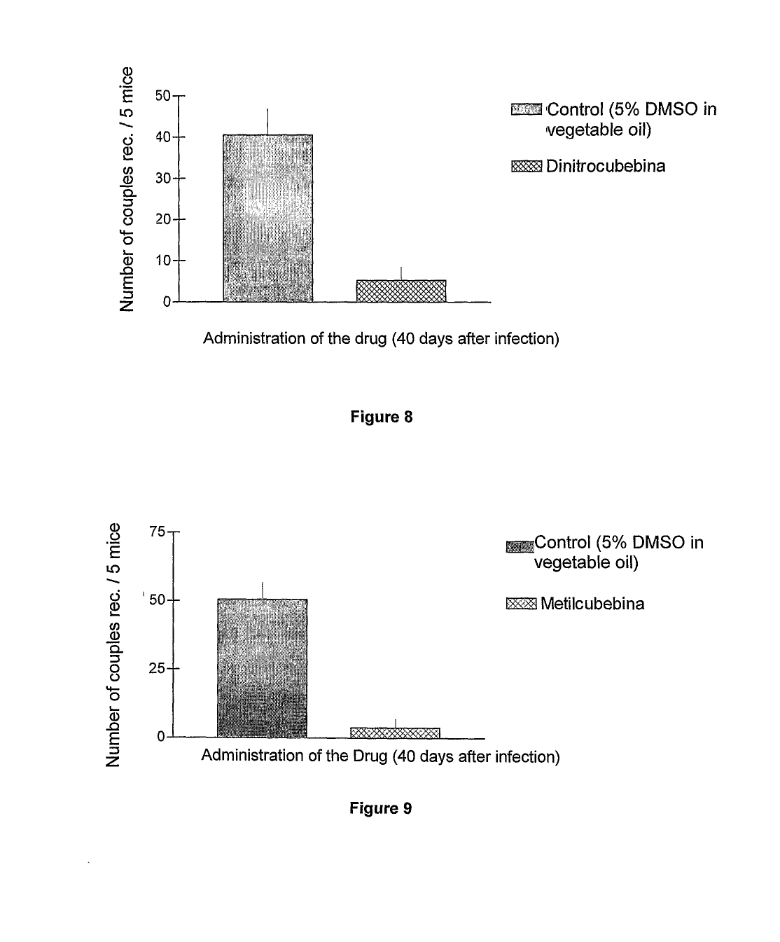

FIG. 8 shows that the number of S. mansoni recovered from BALB/c mice by perfusion of the door-kidney system was about 10%, and that five mice, after 40 days of infection (approximately 200 cercariae), were submitted to test with a single dose of the drug dinitrocubebin (10 mg/kg of weight).

FIG. 9 shows that the number of S. mansoni recovered from BALB/c mice by perfusion of the door-kidney system was about 10%, and that five mice, after 40 days of infection (approximately 200 cercariae), were submitted to test with a single dose of the drug methylcubebin (10 mg/kg of weight).

### EXAMPLES

A medicine containing as its active principle (−)-methylcubebin (6) for treatment.

A medicine containing as its active principle (−)-6,6-dinitroinokinin (8) for treatment.

### Obtained Results:

In in vitro assays, the drugs were active in concentrations varying between 3.5 μg/ml and 50 μg/ml, which shows the potential of this class of substance for the development of new antiparasitic drugs.

In in vivo tests, the drugs (−)—O-methylcubebin (6) and (−)-6,6′-dinitronokinin (8) presented results similar to the obtained with praziquantel, in the same concentration (10 mg/kg of weight), i.e. reduction of approximately 90% of the viable worms, after 40 days of infection with S. mansoni. In other tests, the administration of two doses of the drug (−)-O-methylcubebin (6) (10 mg/kg of weight), after 7 and 21 days of infection, was also highly effective, also causing the reduction of about 90% of the viable worms. Such a result was extremely important and promising, since the drug was given to the parasites when they were still maturing and had not yet reached the evolutive form of adult worms. Therefore, these assays demonstrate that the drug given also acts on the intermediary forms of the parasite (schistosomulus), in the vertebrate host, which is not observed in the praziquantel, which acts only on the evolutive form of adult worms of S. mansoni. Thus, the developed medicine may be used not only as a healing drug, but also as a prophylactic drug in the combat against schistosomosis. Another advantage relates to the low toxicity of these substances for use in antiparasitic therapeutics, since many of these derivatives have hepatic-protecting and antioxidant action. Thus, the use of these lignans certainly offers higher safety for therapeutic use than praziquantel. So, the use of these substances in medicines for longer treatments is safer than with praziquantel and they have nearly no significant side effects. Finally, this group of substances is obtained from natural sources, which is not applicable to praziquantel.

1. Synthetic and semi-synthetic derivatives of dibenzylbutyrolactonic lignans, especially (−)-O-methylcubebin and (−)-6,6′-dinitronokinin obtained by partial synthesis from (−)-cubebin, the derivative having antiparasitic activity in doses having concentrations between 3 and 20 mg/kg of body weight.

2. Process to obtain synthetic and semi-synthetic derivatives from dibenzylbutyrolactonic skeletons, of dibenzocyclooctanic lignans, the process comprising oxidizing the dibenzylbutyrolactonic skeleton by using rhutenium oxide as oxidizing agent in a dichloromethane solution, followed by the use of oxidizing agents for the inclusion of oxygenated groups in the benzyl carbon.

3. Process to obtain derivatives of furofuranic lignans, comprising extracting the derivatives from plant species of the family Rutaceae, especially those that belong to the genus Zanthoxylum.

4. Process to obtain derivatives of aryltetralynic lignans, as well as their synthetic and semi-synthetic derivatives showing substituents in at least one of positions 2, 3, 4 or 5 of the aromatic rings, the process comprising reacting the dibenzylbutyrolactonic skeleton with trifluoroacetic acid in dichlormethane, followed by reaction with DIBAL reagent.

5. Natural derivatives of furofuranic lignans, especially (+)-sesamin (10), piperitol (11), pluviatilol (12) and xantoxilol (13) obtained by the process of claim 3, wherein the derivatives present significant antiparasitic activity.

6. Synthetic and semi-synthetic derivatives of dibenzocyclooctanic lignans, especially stegan (22), steganon (23), steganacin (24) and steganol (25) obtained by the process of claim 2, wherein the derivatives present significant antiparasitic activity.

7. Synthetic and semi-synthetic derivatives of aryltetralinic lignans, especially morelensin (1a) and poligamain (2a) and their derivatives by the process of claim 4, wherein the derivatives present significant antiparasitic activity.

8. Therapeutic method comprising the use of the compounds obtained in claim 1 as active principles for formulations employed in antiparasitic therapeutics.

9. Therapeutic method comprising the use of the compounds obtained in claim 1 as active principles for formulations employed in schistosomocide therapeutics.

10. Use of the compounds described in claim 1 in the formulation of medicines to treat parasitosis.

11. Use of the compounds described in claim 1 in the formulation of medicines to treat parasitosis caused by parasites of the genus Schistosoma.

12. Formulation of antiparasitic medicines containing the compounds obtained in claim 1 for at least one of oral, rectal, vaginal or parenteral administration.

13. Therapeutic method comprising the use of the compounds obtained in claim 5 as active principles for formulations employed in antiparasitic therapeutics.

14. Therapeutic method comprising the use of the compounds obtained in claim 6 as active principles for formulations employed in antiparasitic therapeutics.

15. Therapeutic method comprising the use of the compounds obtained in claim 7 as active principles for formulations employed in antiparasitic therapeutics.

16. Therapeutic method comprising the use of the compounds obtained in claim 5 as active principles for formulations employed in schistosomocide therapeutics.

17. Therapeutic method comprising the use of the compounds obtained in claim 6 as active principles for formulations employed in schistosomocide therapeutics.

18. Therapeutic method comprising the use of the compounds obtained in claim 7 as active principles for formulations employed in schistosomocide therapeutics.

19. Use of the compounds described in claim 5 in the formulation of medicines to treat parasitosis.

20. Use of the compounds described in claim 6 in the formulation of medicines to treat parasitosis.
